(19) United States
(12) Patent Application Publication
(10) Pub. No.: US 2008/0255525 A1
(43) Pub. Date: Oct. 16, 2008

(54) Device for finely dosed discharge of syringes

(76) Inventor: Ahmmed Ziah Taufig, Cologne (DE)

Correspondence Address: DILLER, RAMIK & WIGHT, 7345 MCWHORTER PLACE, SUITE 101, ANNANDALE, VA, 22003, US

(21) Appl. No.: 12/081,253

(22) Filed: Apr. 14, 2008

(30) Foreign Application Priority Data
Apr. 13, 2007 (DE) 20 2007 005 481.5

(51) Int. Cl. A61M 5/315

(52) U.S. Cl. 604/207

(57) ABSTRACT

The device comprises a first holding device (10) which is attached to a syringe barrel (11) of a syringe, and a second holding device (25) which is attached to a plunger barrel (21) of the syringe. Both holding devices are linearly guided relative to each other. One of the holding devices carries a dial gage (31) comprising a rotary shaft (30). Said rotary shaft (30) is in engagement with a toothing (29) of the other holding device. An indicator (32) indicates, with a high resolution, the movement of the plunger rod relative to the syringe barrel such that a highly accurate fine dosing of a liquid discharged from the syringe can be realized.

### FIELD OF THE INVENTION

The invention relates to a device for finely dosed manual discharge of syringes, comprising a syringe holder having a first holding device for engaging a syringe barrel and a second holding device for engaging a plunger rod of the syringe, wherein the first and the second holding device are guided in a manner movable relative to each other.

### BACKGROUND OF THE INVENTION

Syringes normally used in the medical and biological fields as well as in laboratories comprise a scale with the aid of which the injected volume to be administered can be read. The dosing accuracy depends on the mode of use and the reading accuracy, which represents in a limitation when the dosing only allows for a small tolerance and the optical resolution of the viewer is limited.

Further, infusion means comprising an electrical drive are known. Here, a first holding device retains the syringe barrel, and a second holding device is coupled with the plunger rod. Both holding devices are moved relative to each other by spindle drives for the purpose of discharging the syringe. Thus a high adjustment accuracy can be attained, however such injection or infusion devices have a high weight such that they are not suitable for various applications.

### SUMMARY OF THE INVENTION

It is an object of the present invention to provide a device for finely dosed manual discharge of syringes, wherein a high-resolution display device is provided such that dosing with a high accuracy can be performed.

According to the invention, the device is characterized in that at one of the two holding devices a signal transmitter is fastened which shows and optically and/or acoustically indicates the position of this holding device relative to the other holding device.

The device according to the invention projects the movement of the syringe plunger to a display device with the aid of a signal transmitter, said display device being either of analog or digital configuration. The signal can be generated either optically or acoustically. The movement of the syringe plunger and thus the injected volume may be of much higher resolution than in the case of conventional syringes. The resolution to IE or Mouse units is of great importance in particular for toxins. The resolution can be increased up to a factor of 2.5 with the aid of known optical systems, such as lenses. For fully utilizing the fine motor skills of a user, a higher resolution is necessary which is attained with the device according to the invention.

The device can be realized in many different ways. Said device may comprise a dial gage supplied and/or operated by a rotary shaft. In this connection, the term “dial gage” must be interpreted within a broad sense. Said term comprises any display device having a rotary shaft as an input means. The function of the rotary shaft is to roll off one of the holding devices. The smaller the diameter of the rotary shaft, the lager becomes the angular rotation per longitudinal distance covered. Thus a high transmission ratio is attained, wherein a relatively small advance movement of the syringe plunger results in a much larger movement or indication of the dial gage. Another possibility would be to provide a linear potentiometer or a linear induction path at one holding device, while the other holding device carries the signal transmitter which responds in a position-dependent manner to the linear potentiometer and/or the induction path.

If the signal transmitter is a dial gage driven by a measuring shaft, it suffices when the measuring shaft frictionally rolls off the opposite holding device. A higher reliability and accuracy of the measuring result are obtained when the rotary shaft and the measuring shaft, with which said rotary shaft cooperates, comprise teeth meshing with each other. Thus any slip is eliminated.

The device may be provided with an additional gearwheel which may be manually rotated for effecting a very accurate advance movement of the plunger rod relative to the syringe barrel. To some extent the additional gearwheel performs the function of a micrometer screw. It may be left up to the user which advance possibility he wishes to select, namely either pushing forward the plunger rod or rotating the additional gearwheel. The aspect comprising the additional gearwheel can be realized both in the variant according to claim 2 and in the variant according to claim 3.

The invention allows for a very accurate fine dosing during discharge of a syringe and for convenient optical monitoring.

### BRIEF DESCRIPTION OF THE DRAWINGS

Hereunder embodiments of the invention are described in detail with reference to the drawings.

FIG. 1 shows a schematic representation of a first embodiment of the device at the beginning of the discharge process.

FIG. 2 shows the same device as FIG. 1 towards end of the discharge process.

FIG. 3 shows another embodiment of the device at the beginning of the discharge process, wherein the dial gage is fastened to the second holding device.

FIG. 4 shows the embodiment of FIG. 3 towards the end of the discharge process.

FIG. 5 shows a third embodiment of the device comprising an additional gearwheel for fine adjusting purposes.

### DETAILED DESCRIPTION OF PREFERRED EMBODIMENTS OF THE INVENTION

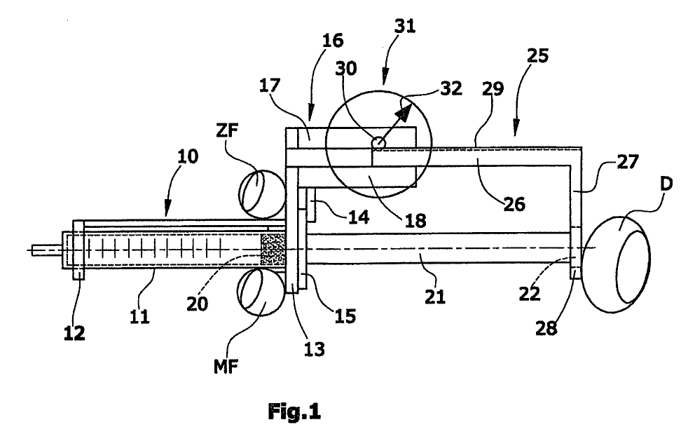

As shown in FIG. 1, a first holding device 10 is provided into which a syringe barrel 11 is inserted such that said syringe barrel is immovable relative to the holding device 10. The holding device 10 comprises a front clip 12 surrounding the syringe barrel, and a rear contact plate 13 having a cantilever arm 14 fastened thereto. Between the contact plate 13 and the cantilever arm 14 a syringe flange 15 is inserted such that the syringe barrel 11 cannot axially move in the first holding device. The contact plate 13 also serves as a finger plate for placing the index finger ZF and the middle finger MF of a hand.

A plunger 20 is movable in the syringe barrel 11, said plunger being connected with a plunger rod 21. At the rear end of the plunger rod 21 a plunger rod flange 22 is located.

The first holding device 10 comprises a guiding device 16 composed of two parallel bars 17,18 for guiding the linear movement of the second holding device 25. The second holding device comprises a holding arm 26, and from the rear end of said holding arm a cantilever arm 27 extends which is fastened to the plunger rod flange 22 via a thumb plate 28 comprising a holding clip. The holding arm 26 comprises a rack-type toothing 29. Said rack-type toothing is in engagement with a toothing provided at a rotary shaft 30. The rotary shaft 30 forms part of a dial gage 31 which is fastened to the first holding device 10. The linear movement of the second holding device 25 relative to the first holding device 10 is converted into a strongly transmitted rotary movement of the rotary shaft 30 such that the indicator 32 of the dial gage shows the advance movement of the plunger 20 with a high resolution.

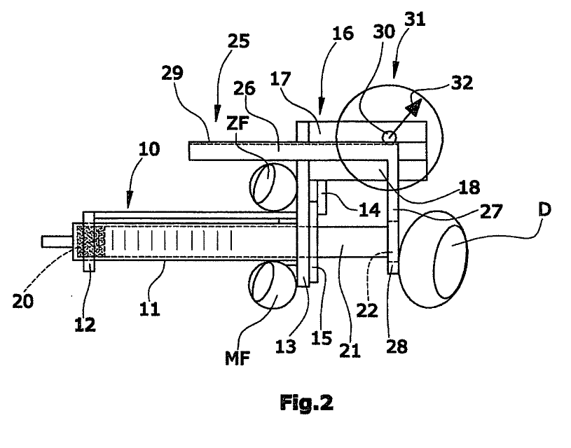

FIG. 2 shows the device of FIG. 1 at the end of the advance movement of the plunger 20. The transmission ratio depends on the diameter of the rotary shaft 30. This diameter may be selected as a very small diameter, wherein the indicator 32 can perform several rotations over the overall adjusting path of the plunger 20.

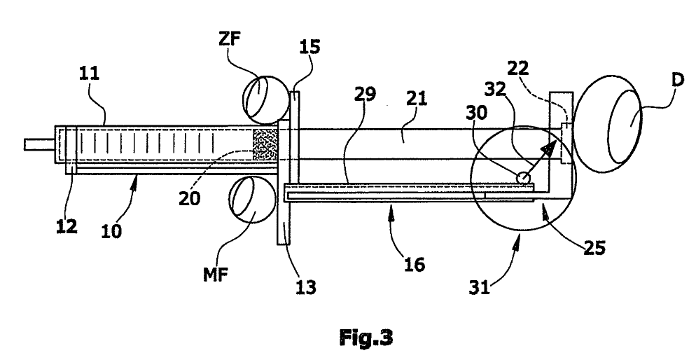

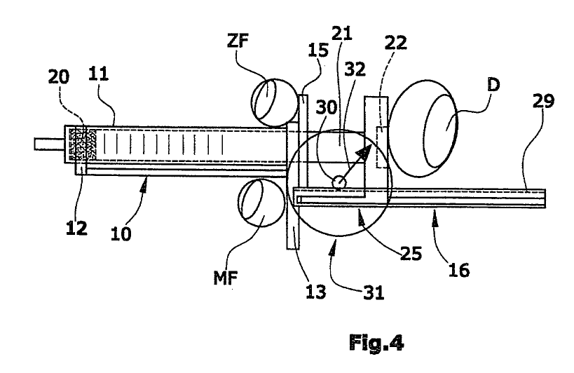

FIGS. 3 and 4 show an embodiment where the first holding device 10 comprises a guiding device 16 far-extending in rearward direction, while the second holding device 25 engaging at the rear end of the plunger rod is relatively short. Here, the dial gage 31 is arranged at the second holding device 25 and moves together with the latter. In this case, a toothing 29 is provided at the guiding device 16 of the first holding device 10, while the rotary shaft 30 is supported at the second holding device 25. The toothing at the circumference of the rotary shaft 30 meshes with toothing 29 of the guiding device 16.

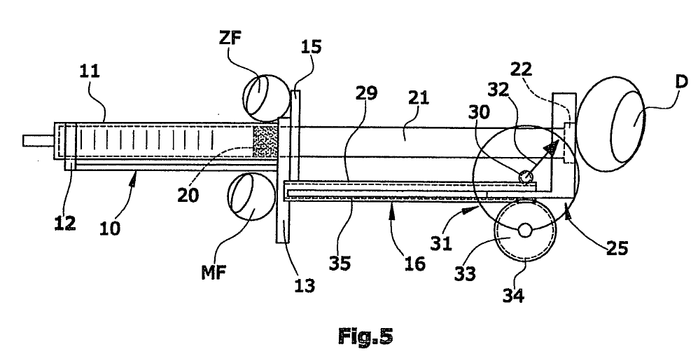

The embodiment shown in FIG. 5 is to a large extent similar to that of FIGS. 3 and 4 such that in the following essentially the differences are elucidated. Here, too, the dial gage 31 as well as the rotary shaft 30 are mounted in the second holding device 25. At the dial gage or the second holding device 25 a gearwheel 33 is supported whose toothing 34 is in engagement with another toothing 35 of the guiding device 16. The gearwheel 33 defines a thumb wheel which rolls off the holding device 25 and pushes forward the plunger rod 21. The transmission of this drive is independent of the transmission of the dial gage.

In the first variant shown in FIGS. 1 and 2, such a drive gearwheel may be provided for advancing the second holding device 25. In this case, the drive gearwheel would be supported at the guiding device 16.

Although the invention has been described and illustrated with reference to specific illustrative embodiments thereof, it is not intended that the invention be limited to those illustrative embodiments. Those skilled in the art will recognize that variations and modifications can be made without departing from the true scope of the invention as defined by the claims that follow. It is therefore intended to include within the invention all such variations and modifications as fall within the scope of the appended claims and equivalents thereof.

What is claimed is:

1. A device for finely dosed manual discharge of syringes, comprising a syringe holder having a first holding device (10) for engaging a syringe barrel (11), and a second holding device (25) for engaging a plunger rod (21) of the syringe, said first and said second holding devices (10,25) being guided in a manner movable relative to each other, wherein at one of said two holding devices (10,25) a signal transmitter is fastened which shows and optically and/or acoustically indicates the position of said holding device relative to said other holding device.

2. The device according to claim 1, wherein the signal transmitter is fastened to the first holding device (10).

3. The device according to claim 1, wherein the signal transmitter is fastened to the second holding device (25).

4. The device according to claim 1, wherein the signal transmitter is a dial gage (31) comprising a rotary shaft (30) rolling off the other holding device.

5. The device according to claim 4, wherein that holding device, where the rotary shaft (30) rolls off, comprises a toothing (29) which meshes with a toothing of said rotary shaft (30).

6. The device according to claim 4, wherein the dial gage (31) comprises an indicator (32) rotating together with the rotary shaft (30).

7. The device according to claim 1, wherein the signal transmitter comprises a display.

8. The device according to claim 1, wherein at one of the holding devices a gearwheel (33) is supported which meshes with a toothing (35) of the other holding device.

9. The device according to claim 1, wherein the first holding device (10) comprises a finger plate (13) and the second holding device (25) comprises a thumb plate (28).
